(19) United States
(12) Patent Application Publication
(10) Pub. No.: US 2026/0294496 A1
(43) Pub. Date: Oct. 1, 2026

(54) HYBRID HEADLESS BONE SCREW

(71) Applicant: Stabiliz Orthopaedics, Inc., Exton, PA (US)

(72) Inventor: Douglas Cerynik, Wynnewood, PA (US)

(73) Assignee: Stabiliz Orthopaedics, Inc., Exton, PA (US)

(21) Appl. No.: 19/631,143

(22) Filed: Mar. 27, 2026

Related U.S. Application Data
(60) Provisional application No. 63/778,916, filed on Mar. 27, 2025.

(51) Int. Cl. A61B 17/86

(52) U.S. Cl. CPC A61B 17/8645; A61B 17/8625; A61B 17/864; A61B 17/866

(57) ABSTRACT

A hybrid headless compression screw includes a distal portion comprising a metallic material and a proximal portion comprising a polymeric material coupled to the distal portion. The metallic distal portion provides high insertion torques and prolonged fragment retention, while the polymeric proximal portion mitigates the risk of hardware prominence associated with all-metallic screws. The polymeric proximal portion may include a biodegradable polymer that degrades and resorbs within the body over time, preventing damage to articular joint surfaces. A drive mechanism transmits rotational force to the distal metallic portion while bypassing the proximal polymeric portion. The proximal and distal portions are coupled by overmolding, mechanical retention features, or other suitable means.

### BACKGROUND

Headless compression screws are designed for articular fixation, particularly in the wrist and foot. Extending this application to other anatomical areas, including the distal femur and femoral head, is of substantial clinical value. The possibility of obtaining solid osteo-chondral fixation with the added benefit of avoiding prominent intra-articular hardware is a novel concept and represents a significant step forward in the repair of these problematic injuries.

All-metallic headless compression screws, while providing high insertion torques and prolonged fragment retention, carry the risk of hardware extending from their insertion point since the larger end portions cannot always compress into the bone. Hardware prominence in such locations may cause damage to the joint surface, pain, and may require additional surgery for removal. Polymer headless compression screws, on the other hand, mitigate hardware prominence but are limited by lower insertion torques and reduced fragment retention capacity compared to their metallic counterparts.

A need exists in the art for a headless compression screw that combines the mechanical advantages of a metallic screw with the biocompatibility and hardware-prominence-avoidance benefits of a polymeric screw. The present invention addresses this need.

### SUMMARY OF THE EMBODIMENTS

The present invention relates to orthopedic fixation devices, and more particularly to headless compression screws comprising a metallic distal portion coupled to a proximal portion comprising a polymeric material.

In one aspect, a hybrid headless compression screw comprises a distal portion comprising a metallic material and a proximal portion coupled to the distal portion, the proximal portion comprising a polymeric material. The metallic distal portion provides for higher insertion torques and prolonged fragment retention, while the polymeric proximal portion mitigates the risk of hardware prominence associated with all-metallic screws.

In certain embodiments, the polymeric proximal portion comprises a biodegradable polymer that is configured to degrade and resorb within the body over time. In other embodiments, the polymeric proximal portion comprises a non-resorbable polymer, including but not limited to polyetheretherketone (PEEK). In further embodiments, the proximal portion may comprise a deformable polymer.

In certain embodiments, the hybrid headless compression screw is cannulated along its length for insertion over a guidewire. A drive mechanism is configured to transmit rotational force to the metallic distal portion in a manner that bypasses the proximal polymeric portion, allowing insertion torques to be directed to the distal metallic portion of the screw.

The proximal portion is coupled to the distal metallic portion by one of a variety of coupling mechanisms, including overmolding, mechanical retention features, snap-fit connections, compression fits, and other means known in the art of materials joining.

In another aspect, the invention provides a system comprising the hybrid headless compression screw and custom instrumentation for preparing the bone and cartilage and inserting the screw.

### BRIEF DESCRIPTION OF THE DRAWINGS

The accompanying drawings, which are incorporated in and constitute a part of this specification, illustrate embodiments of the invention and, together with the description, serve to explain the principles of the invention.

FIG. 1 is a perspective overview of one embodiment of the hybrid headless compression screw, showing the proximal polymeric portion, the distal metallic portion, and a guidewire.

FIGS. 2, 3, and 5 are more detailed view of the hybrid headless compression screw, showing the proximal polymeric portion, threading, and the drive features.

FIG. 4 is a view of the drive polymeric portion engaged with a drive, and the guidewire passing therethrough.

FIGS. 6 and 7 show a detail partial transparent views of the coupling interface between the proximal polymeric portion and the distal metallic portion, showing vertical cone-shaped flutes configured to provide rotational stability and resist vertical translation.

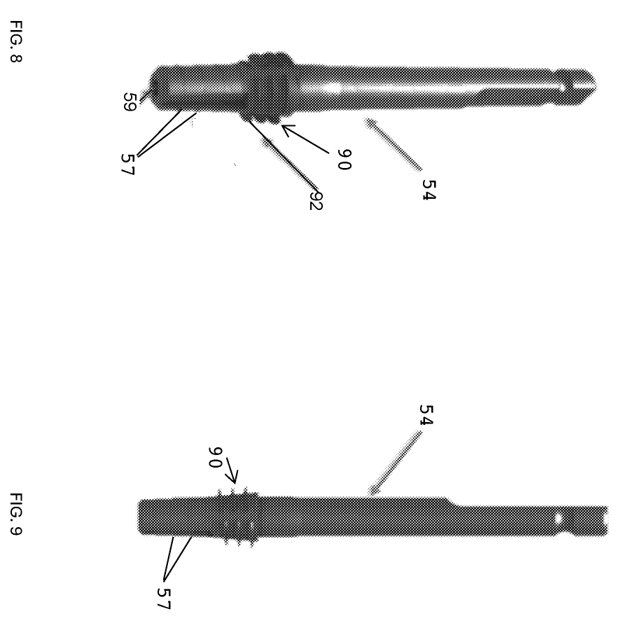

FIGS. 8 and 9 show the engagement between a driver and a tap for use with the hybrid compression screw.

FIGS. 10 and 11 show detents in the metallic base configured to receive the proximal polymeric portion, and the deep drive feature extending into the metallic base.

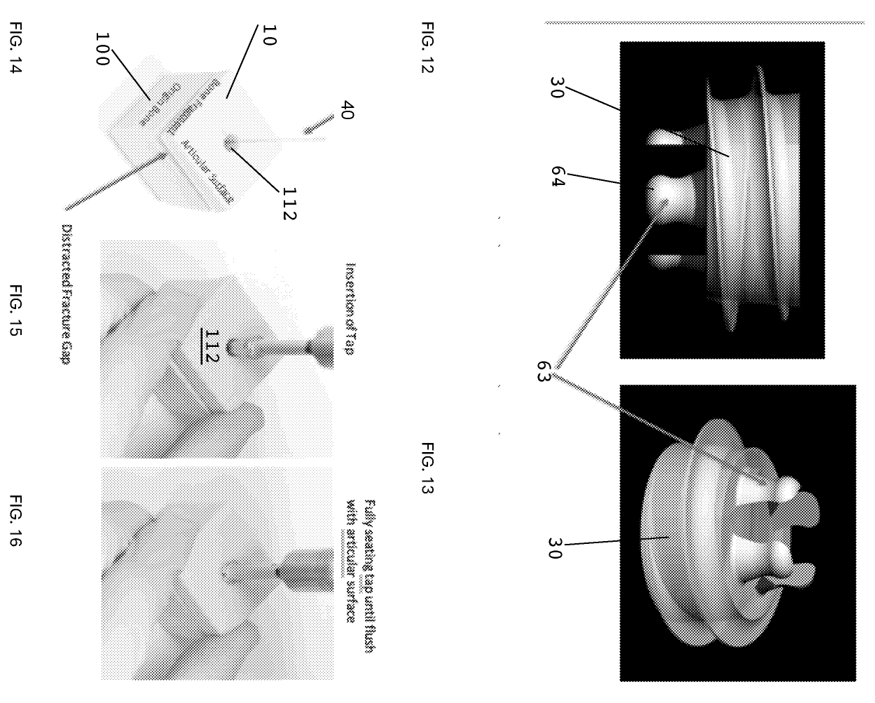

FIGS. 12 and 13 show polymer finger projections configured to mate with detents on the distal metallic portion.

FIGS. 14-20 show the steps of inserting the guidewire, tapping, then engaging the hybrid screw through the fracture.

### DETAILED DESCRIPTION OF THE EMBODIMENTS

The invention herein is not limited to the precise details of construction or operation shown and described, and various modifications and equivalents will occur to those skilled in the art without departing from the spirit and scope of the invention.

### I. Overview

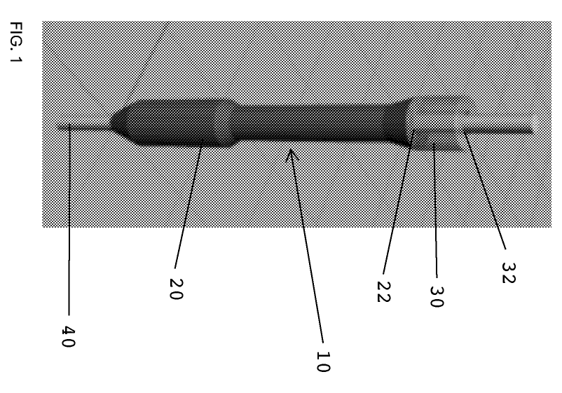

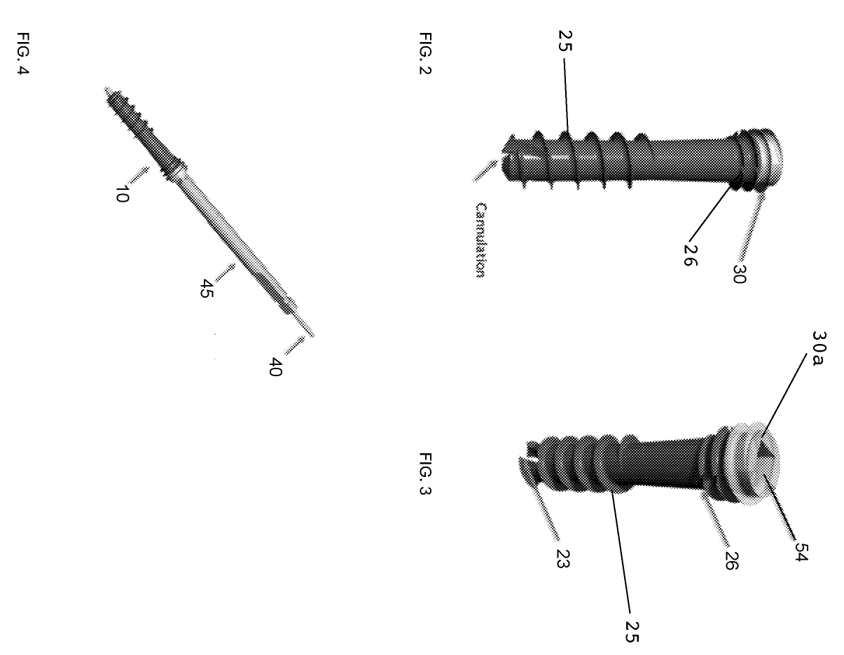

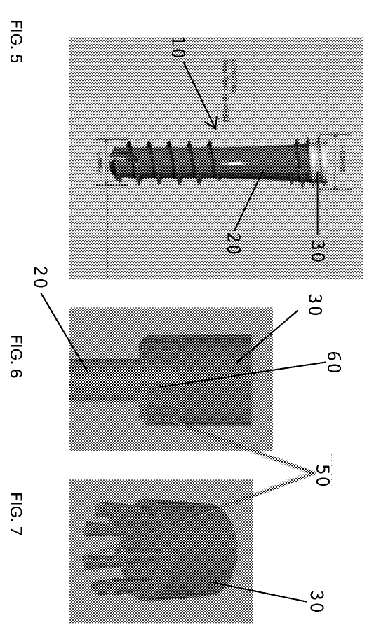

Referring generally to FIGS. 1-5, the hybrid headless compression screw 10 comprises a distal portion 20 comprising a metallic material and a proximal portion 30 coupled to the distal portion, the proximal portion comprising a polymeric material. The hybrid headless compression screw 10 is cannulated along its length to permit insertion over a guidewire 40. The cannulation includes adjacent open channels 22, 32 through the distal portion 20 and proximal portion 30, respectively. FIG. 1 is a model view showing no details thereof, while FIGS. 2-5 show more details.

The metallic distal portion 20 provides for higher insertion torques and prolonged fragment retention compared to all-polymer headless compression screws, where the polymeric head may not be able to withstand the torque required when closing a fracture gap. The polymeric proximal portion 30, however, mitigates the risk of hardware prominence associated with all-metallic screws. Such metallic proud extensions may cause pain or infection, and also may require difficult removal. In contrast, when the hybrid screw proximal end 30 remains proud after insertion, the proximal portion 30 may, in time, degrade and resorb into the body, thereby preventing damage to the joint surface.

The hybrid headless compression screw 10 may be manufactured in any number of external geometries and size offerings. In certain embodiments, the screw is offered in outer diameters of approximately 4.0 mm, 5.0 mm, and 7.0 mm, in lengths ranging from approximately 14 mm to 135 mm in 2 mm and 5 mm increments. The foregoing dimensions are exemplary only.

### II. Distal Metallic Portion

The hybrid compression screw 10 distal portion 20 may be a metallic material. Suitable metallic materials include, but are not limited to, titanium, stainless steel, cobalt chrome, carbon, and combinations and alloys thereof. Additional suitable materials may include calcium phosphates, calcium citrates, silica, magnesium, calcium, and other natural minerals found in bone.

The distal portion 20 includes an insertion cutting thread feature 23 configured to cut bone during insertion of the screw 10, and further distal than the engagement threads 25. In further embodiments, the distal portion 20 includes a reverse cutting thread feature 26 configured to cut bone, in particular bone that may form after insertion, during a later removal of the screw for any reason. The distal portion 20 may be configured to be self-drilling, self-tapping, or both.

The thread pitch and geometry of the distal portion 20 may be configured to produce compression across a fracture site. The distal portion 20 may have a thread pitch differing from the proximal portion 30 to generate a compressive force drawing bone fragments together as the screw advances through the bone across the gap.

### III. Proximal Polymeric Portion

The proximal portion 30 may be made from a polymeric material such as a biodegradable polymer, though it may also be a non-biodegradable, non-resorbable polymer, or a deformable polymer.

Suitable biodegradable polymers include poly(lactic-co-glycolic acid) (PLGA), poly-L-lactic acid (PLLA), polyglycolic acid (PGA), poly(L-lactide-co-D,L-lactide) (PLDLA), polymers containing citrates, and admixtures and combinations thereof. The mechanical and degradation profiles of the biodegradable polymer may be selected in conjunction with a co-development partner based on the intended clinical indication and the anticipated time required for bone healing.

Suitable non-biodegradable polymers include polyetheretherketone (PEEK). Other suitable polymeric materials may include calcium phosphates, calcium citrates, silica, magnesium, calcium, and other natural minerals found in bone.

The proximal portion 30 is expandable upon contact with one or more fluids. In further embodiments, the proximal portion 30 is configured to degrade upon exposure to one or more stimuli, including body fluid, radiofrequency (RF) energy, ultraviolet (UV) light, and combinations of the foregoing.

The proximal head surface portion 30a may be cut, burned, or otherwise trimmed so that the proximal screw surface (an ear below the head portion surface 30a) is flush with the surface of the bone fragment, i.e., the articular joint surface (See FIG. 20, before trimming). This allows the surgeon to precisely tailor the seating of the screw relative to the articular surface.

### IV. Coupling of Proximal and Distal Portions

Referring to FIGS. 5,6, 7, and 10-13, the proximal polymeric portion 30 may be coupled to the distal metallic portion 20 by one of a variety of coupling mechanisms. For example, it may simplest in manufacture to overmold the proximal portion 30 over the distal portion 20.

The proximal portion 30 and distal portion 20 may include two separate components that are joined by snapping, compressing, or otherwise connecting the two components. Such joining mechanisms may include, but are not limited to, dovetails, portals, clearance slots, holes, transverse holes, vertical holes, cone-shaped features, rods, dowels, pins, detents, balls, spring mechanisms.

As shown in more detail in FIGS. 5,6, 7, and 10-13, vertical cone-shaped flutes 50 on the distal metallic portion 20 engage detents 60 in the metallic distal portion 20. Together and so engaged, the flutes 50 and detents 60 provide rotational stability and resist vertical translation of the proximal polymeric portion 30 relative to the distal portion 20. A deep drive feature 54 extends into the metallic base and engages a driver 70 to transmit insertion torque, it being understood that the drive 70 would extend through the open channel 32 in the proximal portion 30 and into the deep drive feature 54.

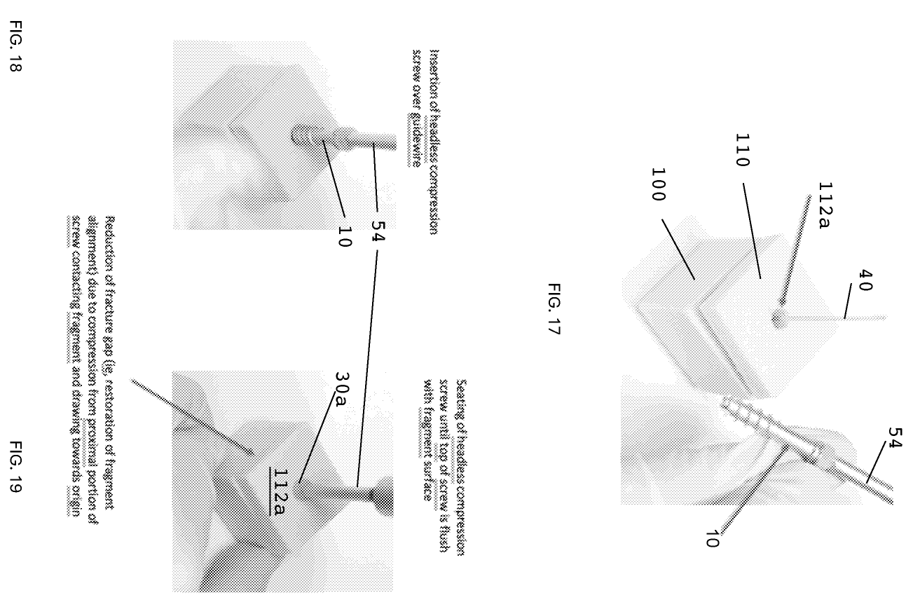

As illustrated in FIGS. 12 and 13, the proximal polymeric portion 30 includes polymer finger projections 63 shaped with bulbous ends 64 that mate with complementary concave portions (not shown) on the distal metallic portion 20. This interlocking engagement is one of the ways to mate the two components and materials.

Retention features may be added to at least one of the proximal portion 30 or the distal portion 20 to aid in reversibly or irreversibly bonding or binding the polymeric material to the metallic material. Such retention features may include, but are not limited to, dovetails, portals, clearance slots or holes, transverse holes, vertical holes, cone-shaped features, rods, dowels, pins, detents, balls, spring mechanisms, and others.

Alternatively, the proximal and distal portions 30, 20 may engage one another through a crimping formed on one or the other that engages the other, though most likely, the crimping would be metal and extend from the distal portion 20. Overmolding the proximal portion 30 onto the distal portion 30 is also possible.

### V. Drive Mechanism

A feature of the hybrid headless compression screw 10 is the drive mechanism 45 that transmits rotational insertion force to the distal metallic portion 20 while bypassing the proximal polymeric portion 30. By routing insertion torques directly to the metallic portion of the hybrid screw 10, the design prevents shear or other mechanical failure of the proximal polymeric portion 30 during insertion.

Said another way, the drive feature 54 extends through the proximal polymeric portion 30 and into the distal metallic portion 20, allowing a cannulated driver to engage the metallic base directly, bypassing the proximal polymer.

### VI. Cannulation and Instrumentation

The hybrid compression screw 10 is cannulated along its length to permit insertion over a guidewire 40. The guidewire 40 facilitates accurate placement of the screw under fluoroscopic guidance.

Custom instrumentation may be used to prepare the bone and cartilage and to insert the screw. In certain embodiments, a cannulated driver engages the drive feature 54 and advances the screw over the guidewire 40. Standard countersinking procedures may be required to properly seat the proximal polymeric portion 30 and avoid damaging its threading.

In certain embodiments, it is anticipated that a replacement screw be recommended should the surgeon remove the screw after the proximal polymeric portion 30 has been inserted, as the polymeric portion 30 may be compromised upon removal.

### VII. Surgical Technique

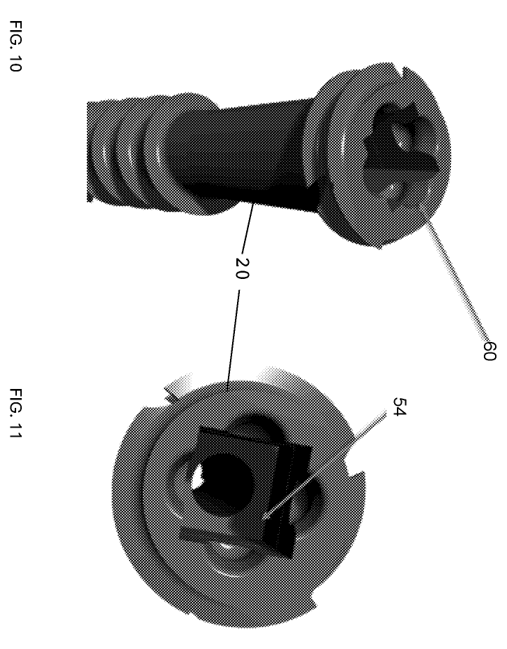

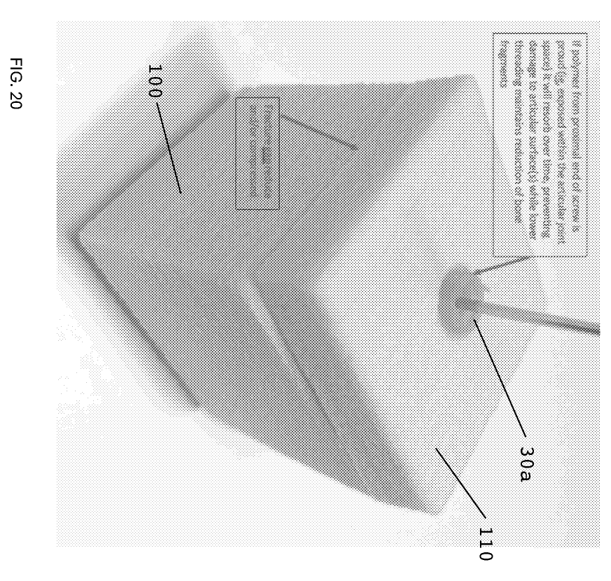

Referring to FIGS. 8, 9, and 14-20, a standard surgical technique for the hybrid headless compression screw 10 adheres closely to the standard techniques for headless screw insertion.

Before following the steps in FIGS. 14-20, FIGS. 8 and 9 show a cannulated tap 90 (cannula 59). The tap may include cutting threads 92 for only a limited portion of its length at its proximal end. These tap cutting threads 92 may engage the threads on the proximal polymeric portion 30 of the hybrid screw 10. The cannulated tap 90 includes a drive cavity (not shown) that engages the driver 45 and also stepped cutting flutes 57 of decreasing diameter along its length that leaves cancellous bone largely unscathed during insertion into the bone.

Moving to the steps, where the origin bone 100 and bone fragment 110 require mending, a surgeon places the guidewire 40 into a pre-drilled hole 112 across the fracture site. The surgeon advances (FIG. 15) the cannulated tap 90 over the guidewire 40 until the tap head is flush with the articular surface 112 (FIG. 16).

The surgeon removes the tap (FIG. 17) from the now-tapped hole 112a, and positions the hybrid headless compression screw 10 for insertion over the guidewire 40. The surgeon then inserts the hybrid screw 10 over the guidewire 40 using the cannulated driver, which engages the drive feature 54 and transmits insertion torque directly to the distal metallic portion 20 (FIG. 18).

As illustrated in FIG. 19, the surgeon advances the screw 10 until the proximal surface 30a of the proximal polymeric portion 30 is flush with the surface of the bone fragment, i.e., the articular joint surface 112a. As illustrated in FIGS. 19 and 20, advancement of the screw 10 draws the bone fragments 100, 110 together, reducing the fracture gap and restoring fragment alignment.

### VIII. Indications

The hybrid headless compression screw system is a single-use device intended for the fixation, correction, or stabilization of small and long bones in adult and adolescent patients. Indications include, by screw size:

For the 4.0 mm screw: fractures of the tarsals and metatarsals; fractures of the olecranon and distal humerus; fractures of the radius and ulna; patella fractures; distal tibia and pilon fractures; fractures of the fibula, medial malleolus, and os calcis; tarso-metatarsal and metatarsophalangeal arthrodesis; metatarsal and phalangeal osteotomies; and osteochondritis dissecans.

For the 5.0 mm screw: fractures of the pelvic ring; small cancellous fragments of the small and long bones; medial and lateral malleolar and pilon fractures; proximal and distal humerus fractures; fractures of the olecranon process; tibial plateau fractures; os calcis, talar, and patellar fractures; fractures of the pelvis and acetabulum; and arthrodesis of the tarsals.

For the 7.0 mm screw: tibial plateau fractures; ankle arthrodesis; calcaneus osteotomies; distal femur fractures; and femoral neck and head fractures.

The above indications are suggestive, and other lengths may be used.

While the invention has been described with reference to the embodiments above, a person of ordinary skill in the art would understand that various changes or modifications may be made thereto without departing from the scope of the claims.

I claim:

1. A headless compression screw (10) comprising:
a distal portion (20) comprising a metallic material, wherein the distal portion includes a drive feature (54) configured to engage a drive mechanism (45) that transmits rotational force to the headless compression screw (10; and
a proximal portion (30) comprising a polymeric material, the proximal portion (30) being coupled to the distal portion (20).

2. The headless compression screw (10) of claim 1, wherein the headless compression screw (10) is cannulated along its length to define a guidewire channel comprising open channels (22, 32) extending through the distal portion (20) and the proximal portion (30), respectively.

3. The headless compression screw (10) of claim 2, wherein the open channel (32) in the distal portion (30) is coaxial with the drive feature (54).

4. The headless compression screw (10) of claim 3, wherein the drive feature (54) extends into the distal portion (20) from a proximal face thereof.

5. The headless compression screw (10) of claim 1, wherein the distal portion (20) comprises engagement threads (25) and an insertion cutting thread feature (23) positioned distal to the engagement threads (25), the insertion cutting thread feature (23) being configured to cut bone during insertion of the headless compression screw (10).

6. The headless compression screw (10) of claim 1, wherein the distal portion (20) further comprises a reverse cutting thread feature (26) configured to cut bone during removal of the headless compression screw (10).

7. The headless compression screw (10) of claim 1, wherein the distal portion (20) is self-drilling, self-tapping, or both.

8. The headless compression screw (10) of claim 1, wherein the distal portion (20) and the proximal portion (30) have differing thread pitches configured to generate a compressive force drawing bone fragments (100, 110) together as the headless compression screw (10) advances through bone.

9. The headless compression screw (10) of claim 1, wherein the proximal portion (30) is coupled to the distal portion (20) by a coupling mechanism selected from the group consisting of overmolding, a snap-fit connection, a compression fit, crimping of the distal portion (20) onto the proximal portion (30), and a mechanical retention feature.

10. The headless compression screw (10) of claim 9, wherein the mechanical retention feature comprises vertical cone-shaped flutes (50) and detents (60) on the distal portion (20) configured to receive and engage the proximal portion (30) to provide rotational stability and resist vertical translation of the proximal portion (30) relative to the distal portion (20).

11. The headless compression screw (10) of claim 9, wherein the proximal portion (30) comprises polymer finger projections (63) having bulbous ends (64) that mate with complementary concave portions on the distal portion (20).

12. The headless compression screw (10) of claim 1, wherein the polymeric material comprises a biodegradable polymer selected from the group consisting of poly(lactic-co-glycolic acid) (PLGA), poly-L-lactic acid (PLLA), polyglycolic acid (PGA), poly(L-lactide-co-D,L-lactide) (PLDLA), polymers containing citrates, and combinations thereof.

13. The headless compression screw (10) of claim 12, wherein the proximal portion (30) is configured to degrade upon exposure to a stimulus selected from the group consisting of body fluid, radiofrequency (RF) energy, ultraviolet (UV) light, and combinations thereof.

14. The headless compression screw (10) of claim 1, wherein the polymeric material comprises a non-biodegradable polymer comprising polyetheretherketone (PEEK).

15. The headless compression screw (10) of claim 1, wherein the proximal portion (30) is expandable upon contact with a fluid.

16. The headless compression screw (10) of claim 1, wherein a proximal head surface (30a) of the proximal portion (30) is configured to be trimmed flush with a surface of a bone fragment.

17. The headless compression screw (10) of claim 1, wherein the metallic material is selected from the group consisting of titanium, stainless steel, cobalt chrome, carbon, and combinations and alloys thereof.

18. A headless compression screw system comprising:
a headless compression screw (10) comprising a distal portion (20) comprising a metallic material and a proximal portion (30) comprising a polymeric material coupled to the distal portion (20); and
a cannulated tap (90) comprising cutting threads (92) at a proximal end thereof and stepped cutting flutes (57) of decreasing diameter along its length, the cannulated tap (90) being configured to prepare a bone hole for receipt of the headless compression screw (10).

19. The headless compression screw system of claim 18, further comprising a driver (45) configured to pass through an open channel (32) in the proximal portion (30) and engage a drive feature (54) of the distal portion (20) to transmit rotational force to the distal portion (20) while bypassing the proximal portion (30).

20. A method of fixing a bone fracture, the method comprising:
placing a guidewire (40) into a pre-drilled hole across a fracture site between an origin bone (100) and a bone fragment (110);
advancing a cannulated tap (90) over the guidewire (40) until the cannulated tap (90) is flush with an articular joint surface (112);
removing the cannulated tap (90) to leave a tapped hole (112a);
inserting a headless compression screw (10) over the guidewire (40), the headless compression screw (10) comprising a distal portion (20) comprising a metallic material and a proximal portion (30) comprising a polymeric material; engaging a driver (45) through an open channel (32) of the proximal portion (30) with a drive feature (54) of the distal portion (20); and
advancing the headless compression screw (10) until a proximal head surface (30a) of the proximal portion (30) is substantially flush with an articular joint surface (112a), thereby drawing the bone fragments (100, 110) together across the fracture site.
